(19) United States
(12) Patent Application Publication
(10) Pub. No.: US 2004/0181183 A1
(43) Pub. Date: Sep. 16, 2004

(54) Bandage based on the teorell-meyer gradient

(76) Inventor: Nicholas A. Sceusa, New York, NY (US)

Correspondence Address: BROWDY AND NEIMARK, P.L.L.C., 624 NINTH STREET, NW, SUITE 300, WASHINGTON, DC, 20001-5303, US

(21) Appl. No.: 10/700,449

(22) Filed: Nov. 5, 2003

Related U.S. Application Data
(60) Provisional application No. 60/453,834, filed on Mar. 12, 2003.

(51) Int. Cl. A61F 13/00

(52) U.S. Cl. 602/48

(57) ABSTRACT

A bandage designed in consideration of Teorell-Meyer gradients is described. This bandage delivers, either individually, or seriatim, pharmaceutically effective amounts of drugs and therapeutic ions to a wound site.

### CROSS-REFERENCE TO RELATED APPLICATIONS

This application claims priority from provisional application No. 60/453,834, filed Mar. 12, 2003, the entire contents of which are hereby incorporated by reference.

### FIELD OF THE INVENTION

The present invention relates to a bandage which delivers, either individually or seriatim, pharmaceutically effective amounts of bioactive agents to a wound site. More particularly, this invention relates to a bandage which is impregnated with a bioactive agent treated in such a way that pH gradient causes the bioactive agent to be driven to a wound site by electrostatic forces. This invention therefore relates to a bandage designed in consideration of naturally occurring pH gradients, known as Teorell-Meyer gradients.

### BACKGROUND OF THE INVENTION

External wounds and concomitant bleeding are common injuries in both civilian and military life. Scratches, cuts, abrasions and the like cause breakage of protective tissue and blood vessels, resulting in the flow of blood out of its normal passageways. This flow of blood washes foreign material out of the wound, and the blood clots to seal the area. Clotting prevents migration of materials into the wound area and into the body of the affected individual. This reduces the likelihood of subsequent infection of the wound.

There are many different treatments for wounds available, most of which involve directly applying pressure to the wounded area and the disposition of an absorptive material or bandage to the wound surface. Direct application of pressure acts to close blood vessels in the area to reduce blood flow; absorb blood flow that is likely to contain foreign material; and to stabilize movement of the blood so that clotting may commence. The disposition of a bandage further absorbs blood flow; provides a barrier to further infection of the wound; and protects the nascent clot while it is still fragile. Ideally, a bandage can also provide antimicrobial or other healing material to the wound surface.

Newer technology for management of wounds includes chemical bandages, polymeric film-forming material applied to the wound area. These products include cyanoacrylate polymers, made with natural coagulants, such as thrombin, prothrombin, and the like. The drawbacks encountered with such formulations, however, include tissue irritation from the cyanoacrylate and the fact that the use of human or animal-derived proteins may be dangerous due to the risk of viral or prion infection, as well as allergic reactions.

For major bleeding incidents, such as those that may be encountered in combat, hemostatic pressure bandages, such as described by Bell, U.S. Pat. No. 5,800,372 can be used to initiate clotting and arrest hemorrhages. However, the collagen used in such dressings is obtained from bone, which may be contraindicated due to the infection risks alluded to above.

Additionally, a hemostatic bandage currently being developed by the Red Cross (but not yet approved by the FDA) has the drawback that it may trigger allergic reactions. This bandage also uses human blood proteins, thus taxing an already overburdened blood supply. It also lacks durability.

Other developments include a chitosan bandage has been developed which uses shrimp cell chitin (Pusateri et al., Journal of Trauma, January 2003, abstract attached). This bandage is reported as being superior to conventional gauze preparations.

Once a wound has been treated, there may be a continuing need to apply medication during the healing process. Presently, such a need is met by continual wound maintenance involving cleaning, debridement where needed, administration of medication and re-bandaging. This process may cause discomfort, time and expense, and may result in inefficient or impaired healing.

Thus, there is a need in the art for a bandage that swiftly provides an anticoagulant when needed in the case of severe bleeding or hemorrhage. It would be further desirable if the same bandage was also capable of continuously delivering medication to the wound during the healing process.

### SUMMARY OF THE INVENTION

It is an object of the present invention to overcome the aforesaid deficiencies in the prior art.

It is another object of the invention to provide a bandage impregnated with a bioactive agent.

It is yet another object of the invention to provide a bandage capable of delivering a cocktail of bioactive agents, wherein said bioactive agents have different migration rates so that the bioactive agents are delivered when needed, at different times in the course of wound management.

A further object of the invention is to provide a method of treating both minor and major wounds, by applying a bandage impregnated with one or more bioactive agents, depending on need.

### DETAILED DESCRIPTION OF THE INVENTION

A new bandage for treatment of wounds is disclosed. Said bandage can administer, either individually or seriatim, bioactive agents to the site of a wound, using charge as a driving principle. Such a bandage is based on the Teorell-Meyer gradient and is a complete departure from conventional wound dressings.

The bandage is impregnated with one or more bioactive agents and will be able to move either cations or anions by taking advantage of naturally occurring concentration gradients. By manipulation of the pH of the bioactive agents to a suitable extent, by using a dosage form buffered at a correct pH, the bioactive agent will be moved electro-osmotically in accordance with Teorell-Meyer flux gradients.

The design of bandages according to this invention that are capable of moving bioactive agents into a wound site in a pH dependent manner, derives mathematically from the Teorell-Meyer Theory. See, Teorell, T., Discussions Faraday Soc., 1956, 21(9), 305-369. The derivation according to this invention predicts that a dosage form buffered at the correct pH will be able to move either the desired positive or negative ions from compartment A to compartment B in an pH dependent osmo-electrophoretic manner, provided a flux gradient exists between two compartments, viz., the compartment of the impregnated bandage pad and the wound site.

Teorell-Meyer dosage forms depend upon bioelectricity for their function. A biologically closed electric circuit (BCEC) is physiologically analogous to an ordinary electric circuit, except that ions, predominantly, as well as electrons, move along and through the circuit. In biological material, the co-transport of electrons occurs in short redox steps. Ions are transported electro-osmotically. Concentration, and consequently, electrical gradients, are maintained by Donnan Equilibria, large sheets of charge in the tissue proteins, and by ion pumps functioning at the expense of ATP. The second half of the circuit, the return half, takes place via passive or facilitated diffusion. Ions will follow, or respond to the flow of current according to their net charge, from one are of charge density to another area of different charge density, as part of the usual BCEC circulation. The local viscosity, and the electrical path length, which is a vector quantity, plays an important role. Vectors have the properties of force, distance (length), according to the gradients that compose them. Controlling the electrical vector makes it possible to control the ion, because the electrical vector is very many times stronger than any of the other which act.

Although a BCEC is electrically closed, it is thermodynamically and physiologically open, which makes it possible to place a dosage form in a predetermined location. This property is used to artificially induce a gradient, using appropriate buffering, companion, and carrier molecules. Certain molecules may act as all three at the same time, and the amino acids and their congeners are ideal for this purpose. By introducing the specially designed and buffered dosage form, the pH of the recipient compartment, in which the form is placed, is changed relative to the target compartment, setting up the induced gradient and corresponding concentration cell. This is provided for by the Lewis acid-base definition, which considers all positive charges as acids and all negative charges as bases.

Inducing the pH change and controlling the bioelectrical field and corresponding electrical vector makes it possible to manipulate the direction of ionic flow and transport. Since the electrical vector is many times more powerful than the other vectors acting, the ionic glow can be stopped or reversed for the time the induced field is present. If the electrical vector is coupled to act in the same direction as the other vectors, the effect is most powerful. The three vectors which are known to act are the hydrostatic vector, the particulate (colligative) vector, and the electro-motive force (electro-osmotic) vector.

It should be remembered that the association constant (Ka) and its reciprocal, the dissolution constant, Kd, for any complex are pH dependent. In the context of an electrical gradient inside a concentration cell, these constants may also be considered to be electrically dependent. In other words, at one pH a complex may be completely associated, and at another pH, almost completely dissociated.

Therefore, for any given complex, the concentration cell has a continually changing spectrum of pH and association constants inherent within it. This change over distance, which operates most strongly at the endpoints, permits the system to deliver ions in the way it does.

Charged particles do not easily penetrate membranes, because charged particles are generally not lipid soluble. This is generally true, but is not universal. If a particle is fairly small and its charge comparatively large, and the membrane relatively thin, an ion will be dragged through the lipid bi-layer membrane. By arranging the electrical vector in the same direction as the other diffusion vector, this penetration can be greatly improved. This is particularly useful for ions delivered perpendicular to membranes, such as the thin membranes of the nasal conchae in the nose.

Therefore, a bandage of the present invention is ideal for use in therapeutically targeting a wound site and will provide more direct application of a bioactive agent to a target wound site than most conventional wound dressings and methods of treatment, particularly those that must rely on manipulations of the dressing and sensitive wound site such as cleaning, debridement, application of topical therapeutics and rebandaging. Such advantages allow for the impregnated bandage to actually contain a lower dosage of bioactive agent, since a higher percentage of drug is delivered to the target area. The drug can also be delivered directly to the target area as needed.

Furthermore, the agent in the bandage can be targeted to specific areas under the dressing according to the prevailing Donnan Equilibrium of that tissue. These equilibria can be mapped and may differ between traumatized and nontraumatized skin and other body surfaces (e.g. mucosa) due to a variety of factors.

Said bandage may be impregnated with almost any therapeutic agent that is capable of existing in ionized form, although those agents of lower molecular weight or size will be transported faster and are therefore preferred. Non-ionic agents require an ionizable carrier, which must meet the further requirements of providing for favorable release of the drug at the target site as well as being metabolizable or otherwise easily eliminated physiologically.

In the language of the Teorell-Meyer gradient, the bandage, which forms a repository compartment will provide the bioactive agent needed to treat a wound site into a recipient compartment, based on the Teorell-Meyer gradient of differing pHs between the two compartments. Use of the bandage entails determining the pH of each compartment, and can be applied to compartments that are adjacent or contiguous, or that are separated only by a thin membrane. The repository compartment is in the form of a bandage containing the desired bioactive agent.

The term “bandage” is intended herein to encompass any material disposed upon or inside the body for medical or therapeutic purposes, including wound and surgical dressings, drapes, bandages, pads, gauze, tampons, sponges and the like.

It is expected that a medical or pharmaceutical practitioner of ordinary skill in the art would appreciate the full range of applicability of the invention.

Preparation of the wound dressing is carried out with an eye towards the type of contiguous recipient compartment system to which this invention applies. Clearly, the recipient compartment is the wound surface, which is composed of compromised skin and the underlying compromised tissue. This preparation of the dressing must be dictated largely by pH differences between the two compartments, although other factors may be present as well. Generally, a difference of at least 0.1 pH units between the compartments is necessary, although the larger the pH difference the faster the bioactive agent will be transported. A pH difference of 2.0 pH units is usually preferred, but a larger difference is possible according to the tolerance of the tissues. Thus, each individual bioactive agent- or agents-impregnated bandage has its own limits based on the practical pH difference between the compartments and each bandage should be prepared according to the desired transport time that makes sense for the system.

The bioactive agent or agents must also be selected. Transfer using the impregnated bandage is applicable to almost any drug that is in anionic, cationic or ionizable form. Ionic drugs should be hydrated. Non-ionic drugs may also be used as they can be released from an ionizable carrier such as cyclic carbohydrates and cyclodextrans. The speed of travel of the drug depends on the charge, the atomic or molecular diameter, the molecular weight and the viscosity of the medium in which it travels. The bandage will move any ionic substance with a molecular weight of up to thousands of Daltons.

In the case of a cationic (positively charged) or acid drug, the repository compartment (the bandage) must have an induced pH substantially lower that the recipient compartment (the wound site). Conversely, for an anionic (negatively charged) or basic drug the repository compartment must have an induced pH higher than the recipient compartment. Thus, the selection of the buffering system for the dosage form is highly significant. The range of buffers employed correspond to the range of pHs found in the human body, the lowest pH presently known is that of the stomach which is about pH 0.1, the highest pH presently known is about 9.0 and is found in the lower intestine. Untraumatized human skin generally has a pH around 5.5-6.0. The buffer or buffer system must last long enough for consumption of the entire dose for complete drug transport to occur.

While the buffers selected must create a pH differential between the compartments of ideally 2.0 pH units or more to cause rapid drug movement, greater or smaller pH differences are not beyond the scope of this invention. When selecting the buffer, physiological considerations must also be taken into account, viz., the amount of pH difference between the dosage buffer and the repository compartment that the tissue of that compartment will tolerate. One skilled in the art can readily formulate a medicament having the requisite pH without undue experimentation.

For the purpose of this invention, the 20 physiologically accepted amino acids and their congeners (e.g., orotic acid, carnitine, ornitine) are generally preferred. The buffers systems usually contain at least two components: a salt and its correlative acid, or base. Buffers may be single compounds in certain cases, such as solutions of amino acids, Tris®, and other compounds containing both acid and basic groups on the same molecule. A buffering system may be complex, containing several components. It may also contain non-related salts and amino acids or similar zwitterionic compounds.

The buffering agent should be able to reliably buffer at the chosen pH, which may be anywhere within the physiological range, so as to preferably maintain a difference of at least 2 pH units between the repository and recipient compartments, according to tissue tolerance, for the preferred embodiment of the invention, to exert substantial buffering capacity within this range. Preferred buffering agents are the amino acids, hydrogen and dihydrogen phosphates, such as sodium dihydrogen phosphate and mixtures of sodium dihydrogen phosphate with sodium hydrogen phosphate, calcium tetrahydrogen phosphate, citric acid and mixtures of citric acid and its monosodium salt, fumaric acid and its monosodium salt, adipic acid and its monosodium salt, tartaric acid and its monosodium salt, ascorbic acid and its monosodium salt, glutamic acid, aspartic acid, betaine hydrochloride, hydrochlorides of amino acids, such as arginine monohydrochloride and glutamic acid hydrochloride and saccharic acid, and other suitable GRAS ingredients herein incorporated by reference.

As discussed supra, hydro-osmotic pressure, concentration and pH differences between a bioactive agent or agents-impregnated bandage and a wound site form a Teorell-Meyer flux gradient. A Teorell-Meyer flux gradient occurs if there is a two or more compartment unit in which different concentrations, relative charges, and hydro-osmotic pressure exist. There may be one or more ionic substances or electrolytes present, and the method is dependent on total relative force rather than any single element. Thus, the driving force for this dosage form depends on the sum of three vector force components: chemical and electrical force and hydro-osmotic pressure, as comprehensively detailed in U.S. Pat. No. 6,414,033, herein incorporated by reference in its entirety.

To summarize, in the practice of this invention, therefore, the following steps must be observed. To move a positively charged (i.e., acid) ion, drug or pro-drug from the bandage to the wound site, the bandage pH must be lowered below that of the target or destination area for the drug, i.e., the site of the wound to be treated. Conversely, to move a negatively charged (Le., basic) drug, the pH of the dressing is raised above that of the wound site. This movement is osmo-electrophoretic, and the energy is supplied by the Teorell-Meyer concentration gradient between the bandage and the wound site. Using this bandage as applied to treatment of wounds, almost any FDA or homeopathically approved bioactive agent may be used to impregnate the bandage. The identification of said agents will be apparent to one of ordinary skill in the art.

As used herein, the term “bioactive agent” is identical to the meaning of the term “drug” employed in the 26th Edition of Stedman's Medical Dictionary, viz., “[a] [t]herapeutic agent; any substance, other than food, used in the prevention, diagnosis, alleviation, treatment, or cure of disease.” In addition, for the purposes of the present invention, a bioactive agent may be any substance that affects the activity of a specific cell, bodily organ or function. It may be an organic or inorganic chemical, a biomaterial, etc. Any chemical entity of varying molecular size (both small and large) exhibiting a therapeutic effect in animals and humans and/or used in the diagnosis of any pathological condition, including substances useful for medical imaging such as fluorescent dyes and radioactive isotopes fits the above definition.

### EXAMPLE

Based on the above discussions, a bandage is formulated to be placed on the skin of a wounded individual. Said bandage staunches an active flow of blood by both the application of mechanical pressure and, optionally, depending on wound severity, a concomitant release of a clot-promoting compound such as, without limitation, thrombin, fibrinogen, enzymes such as factor Xa (FXa) and/or factor VII (FVIIa), and the like. In engineering this bandage, the pH of the recipient compartment, i.e., the wound, must be considered.

In the case of less severe wounds, a single agent such as an antibiotic may be delivered by the bandage, to promote healing. Other examples of treatment using bandages according to the present invention are burns or eruptions of the skin. Bandages according to the present invention can be used wherever there is a recipient compartment for delivery of the active ingredient (S).

The foregoing description of the specific embodiments will so fully reveal the general nature of the invention that other can, by applying current knowledge, readily modify and/or adapt for various application such specific embodiments without undue experimentation and without departing from the generic concept. Therefore, such adaptations and modifications should and are intended to be comprehended within the meaning and range of equivalents of the disclosed embodiments.

It is to be understood that the phraseology or terminology employed herein is for the purpose of description and not of limitation. The means and materials for carrying out various disclosed functions may take a variety of alternative forms without departing from the invention.

Thus, the expressions “means to . . . ” and “means for . . . ” as may be found in the specification above and/or in the claims below, followed by a functional statement, are intended to define and cover whatever structural, physical, chemical, or electrical element or structures which may now or in the future exist for carrying out the recited function, whether or nor precisely equivalent to the embodiment or embodiments disclosed in the specification above. It is intended that such expressions be given their broadest interpretation.

As will be apparent to one skilled in the art, various modifications can be made within the scope of the aforesaid description. Such modifications being within the ability of one skilled in the art form a part of the present invention by the appended claims.

What is claimed is:

1. A bandage impregnated with a bioactive agent which delivers a pharmaceutically effective amount of said bioactive agent to a wound site wherein a pH difference between the bandage and the wound site drives delivery of the bioactive agent into the wound site.

2. The bandage of claim 1, wherein the bioactive agent is a clot-promoting agent.

3. The bandage of claim 1, wherein the bioactive agent is an antibiotic.

4. A bandage impregnated with multiple bioactive agents, wherein said bioactive agents have different pH's and different migration rates so that the agents are delivered in pharmaceutically effective amounts, when needed, to a wound site, at different times in the course of wound management, whereby pH difference between the bandage and the wound site drives delivery of the bioactive agent into the wound site.

5. The bandage of claim 4, wherein one of the bioactive agents is a clot-promoting agent.

6. The bandage of claim 4, wherein one of the bioactive agents is an antibiotic.

7. A method for treating a wound, the method comprising:
applying a bandage impregnated with a bioactive agent which delivers a pharmaceutically effective amount of said bioactive agent to a wound site wherein a pH difference between the bioactive agent bandage and the wound site drives delivery of the bioactive agent into the wound site.

8. The method of claim 7, wherein the bioactive agent is a clot-promoting agent.

9. The method of claim 7, wherein the bioactive agent is an antibiotic.

10. A method for treating a wound, comprising:
applying a bandage impregnated with multiple bioactive agents, wherein said bioactive agents have different pH's and different migration rates whereby the agents are delivered in pharmaceutically effective amounts, when needed, to a wound site, at different times in the course of wound management, wherein a pH difference between the bandage and the wound site drives delivery of the bioactive agent into the wound site.

11. The method of claim 10, wherein one of the bioactive agents is a clot-promoting agent.

12. The method of claim 10, wherein one of the bioactive agents is an antibiotic.
